(19) United States
(12) Patent Application Publication
(10) Pub. No.: US 2022/0412891 A1
(43) Pub. Date: Dec. 29, 2022

(54) Detection device, method for preparing the same, detection system comprising the same, and detection method using the same

(71) Applicant: MAN Technology Co., Ltd., Tainan City (TW)

(72) Inventors: Jiunn-Der LIAO, Tainan City (TW); Chia-Wei CHANG, Tainan City (TW)

(21) Appl. No.: 17/359,761

(22) Filed: Jun. 28, 2021

(51) Int. Cl. G01N 21/65; C12Q 1/04; B21D 37/01

(52) U.S. Cl. CPC G01N 21/658; C12Q 1/04; B21D 37/01; G01N 2201/06113

(57) ABSTRACT

A detection device for virus detection is provided, which includes: a carrier including a recess; and a metal layer disposed in the recess and having a contact angle ranging from 0 degrees to 10 degrees, wherein a plurality of cavities are formed on a first surface of the metal layer opposite to a second surface of the metal layer facing the carrier, the plurality of cavities are arranged in an array, and a plurality of first protrusions are formed on the first surface of the metal layer and near to the plurality of cavities. In addition, a detection system for virus detection comprising the aforesaid detection device, a method for detecting viruses using the aforesaid detection device, and a method for preparing the detection device are also provided.

### BACKGROUND

### 1. Field

The present disclosure relates to a detection device, a method for preparing the same, a detection system comprising the same and a detection method using the same. More specifically, the present disclosure relates to a detection device for virus detection, a method for preparing the same, a detection system comprising the same, and a detection method using the same.

### 2. Description of Related Art

Rapid and accurate detection and identification of a dangerous pathogen is extremely helpful for a diagnosis or therapy of an early-stage disease caused by the pathogen. Currently, antibody-based bioarrays can be employed to monitor viruses and such techniques are, for example, enzyme-linked immunosorbent assays (ELISA), fluorescent antibody arrays, and serological testing. However, these techniques may meet some clinical problem such as being time-consuming, false positive, etc.

In recent years, as polymerase chain reaction (PCR) keeps advancing, related methods such as single-nucleotide polymorphism (SNP) in which a target DNA sequence is also proliferated, become alternative approaches to virus identification. However, complex purification and isolation steps of the target DNA are required to be performed prior to ELISA or PCR. Therefore, if such complex purification and isolation steps can be eliminated, identification of a target virus can be simplified and accelerated.

At present, in virus identification or detection, the major difficulties are associated with the complex purification and isolation, the detectable amount, apparent size, and dimension of virus, and the requirement of chemical identification databases for specific viruses. In general, rapid-screening detection is difficult to achieve at an early stage of virus infection since the virus amount often is unable to reach the detectable amount, more than 10⁶ plaque-forming units (PFU)/ml, in biomolecular assays where the purification procedure is employed. The reagents used in common biomolecular assays are far smaller than an apparent size and dimension of a virus. Thus, only a portion of the integral virus can be detected, resulting in incomplete virus information. Also, it is difficult to establish chemical identification information for the integral virus in a database.

Biomolecules or their fragments can be investigated by resonating mechanical cantilevers, evanescent wave biosensors, or atomic force microscopy, but these approaches are used only to measure the amount of the biomolecules. Alternatively, a labeling method can be employed to detect microorganisms by a particular functional group. However, it is difficult to prepare samples without contamination, or easy to overlap between signals of the labeling functional group and the target microorganism. Hence, these techniques consume a lot of time for detection and cannot meet a clinical requirement for rapid detection.

### SUMMARY

The object of the present disclosure is to provide a detection device, which can be applied to surface-enhanced Raman scattering (SERS) technology for virus detection.

The detection device of the present disclosure comprises: a carrier comprising a recess; and a metal layer disposed in the recess and having a contact angle (θ) ranging from 0 degrees to 10 degrees (for example, 0°≤θ≤10°, or 0°<θ<10°). Herein, a plurality of cavities are formed on a first surface of the metal layer opposite to a second surface of the metal layer facing the carrier, the plurality of cavities are arranged in an array, and a plurality of first protrusions are formed on the first surface of the metal layer and near to the plurality of cavities.

In the detection device of the present disclosure, the surface of the metal layer is formed with cavities arranged in an array. When the detection device of the present disclosure is used with a Raman spectrometer to detect the virus sample, the SERS effect can be enhanced, and it is possible to detect viruses at very low concentration. Furthermore, in the detection device of the present disclosure, the metal layer has a low contact angle. When the virus sample is applied onto the metal layer of the detection device of the present disclosure, the virus sample can adhere onto the metal layer well due to the hydrophilicity of the metal layer, and thus the virus particles in the virus sample can be well captured by the cavities of the metal layer.

The detection device of the present disclosure may selectively comprise a substrate. In one aspect of the present disclosure, the detection device comprises the substrate, wherein the substrate is disposed in the recess and the metal layer is disposed on the substrate. In another aspect of the present disclosure, the detection device does not comprise the aforesaid substrate, and the metal layer is directly disposed in the recess.

In the present disclosure, the material of the carrier or the substrate is not particularly limited, and may include, for example, quartz, glass, silicon wafer, sapphire, polycarbonate (PC), polyimide (PI), polypropylene (PP), polyethylene terephthalate (PET) or other plastic or polymer material, or a combination thereof, but the present disclosure is not limited thereto.

In the present disclosure, each of the plurality of cavities may have a shape of an inverted polygonal pyramid, and the shape of the cavities can be selected according to the viruses to be detected. The inverted polygonal pyramid may be, for example, an inverted triangular pyramid, an inverted rectangular pyramid, an inverted pentagonal pyramid or an inverted hexagonal pyramid.

In the present disclosure, when the cavities have the shapes of the inverted polygonal pyramids, each of the plurality of cavities has a bottom point, and a distance between the bottom points of two adjacent cavities of the plurality of cavities may be in a range from 100 nm to 1000 nm, for example, 100 nm to 950 nm, 100 nm to 900 nm, 150 nm to 900 nm, 150 nm to 850 nm, 200 nm to 850 nm, 200 nm to 800 nm, 250 nm to 800 nm, 250 nm to 750 nm, 300 nm to 750 nm, 350 nm to 750 nm, 400 nm to 750 nm, 450 nm to 750 nm, 500 nm to 750 nm, 500 nm to 700 nm or 520 to 700 nm. Herein, the distance between the bottom tips of the two adjacent cavities can be selected according to the viruses to be detected.

In the present disclosure, when the cavities have the shapes of the inverted polygonal pyramids, each of the plurality of cavities may have a plurality of inclined surfaces, and one of the plurality of first protrusions connects to one of the plurality of inclined surfaces. For example, when the cavity has the shapes of the inverted triangular pyramid, the cavity has a triangular opening with three sides which respectively connects to one of the first protrusions, and thus one first protrusion connects to one inclined surface at each side of the triangular opening.

In the present disclosure, each of the plurality of cavities may have a width to depth ratio ranging from 3:1 to 5:1 measured from the first surface of the metal layer. In one embodiment of the present disclosure, the width to depth ratio may be about 5:1.

In the present disclosure, each of the plurality of cavities may have a depth ranging from 20 nm to 300 nm measured from the first surface of the metal layer, for example, 20 nm to 280 nm, 20 nm to 250 nm, 30 nm to 250 nm, 30 nm to 230 nm, 40 nm to 230 nm, 40 nm to 200 nm, 45 nm to 200 nm, 45 nm to 180 nm, 50 nm to 180 nm, 50 nm to 150 nm, 55 nm to 150 nm, 60 nm to 150 nm, 65 nm to 150 nm, 70 nm to 150 nm, 75 nm to 150 nm, 80 nm to 150 nm, 85 nm to 150 nm, 90 nm to 150 nm, 95 nm to 150 nm, 95 nm to 140 nm or 100 nm to 140 nm. Herein, the depth of each of the cavities can be selected according to the viruses (for example, the size of the virus particle) to be detected.

In the present disclosure, each of the plurality of cavities may have a width ranging from 100 nm to 1400 nm measured from the first surface of the metal layer, for example, 100 nm to 1200 nm, 100 nm to 1100 nm, 200 nm to 1100, 200 nm to 900 nm, 300 nm to 900 nm or 300 nm to 700 nm. Herein the width of each of the cavities may be selected according to the viruses (for example, the size of the virus particle) to be detected. Herein, the width of the cavities can be the length of the sides of the opening of the cavities.

In the present disclosure, each of the first protrusions has a height, which can be 10% to 20% of the depth of the cavity near to the first protrusion. In the present disclosure, each of the plurality of first protrusions may have a height ranging from 2 nm to 60 nm measured from the first surface of the metal layer, for example, 2 nm to 55 nm, 2 nm to 50 nm, 3 nm to 50 nm, 3 nm to 45 nm, 4 nm to 45 nm, 4 nm to 40 nm, 4.5 nm to 40 nm, 4.5 nm to 35 nm, 5 nm to 35 nm, 5 nm to 30 nm, 5.5 nm to 30 nm, 5.5 nm to 28 nm, 6 nm to 28 nm, 6 nm to 27 nm, 6.5 nm to 27 nm, 6.5 nm to 26 nm, 7 nm to 26 nm, 7 nm to 25 nm, 7.5 nm to 25 nm, 7.5 nm to 24 nm or 8 nm to 24 nm. Herein, the height of each of the first protrusions can be selected according to the viruses (for example, the size of the virus particle) to be detected. When the first protrusions are formed near to the cavities, the viruses particles can be more easily entrapped into the cavities, and thus the SERS detection effect can further be improved.

In the present disclosure, each of the first protrusions has a width ranging from 270 nm to 500 nm measured from the first surface of the metal layer. Herein, the width of the first protrusions is the maximum width of the first protrusions.

In the present disclosure, the detection device may further comprise a cover plate disposed on the metal layer. The cover plate can protect the nano-array structures (i.e. the cavities and the first protrusions) on the metal layer. Here, the cover plate should have high transmittance, so the incident laser provided by a Raman spectrometer can penetrate the cover plate and achieve to the metal layer. In the present disclosure, the transmittance of the cover plate may be more than 89%, for example, ranged from 89% to 99%, 89% to 95% or 90% to 95%. Herein, the material of the cover plate is not particularly limited as long as the cover plate has high transmittance. For example, the material of the cover plate may include polymethyl methacrylate (PMMA), other plastic or polymer material, or a combination thereof, but the present disclosure is not limited thereto.

In the present disclosure, the carrier may further comprise a first channel, the cover plate may comprise a sample inlet, and the sample inlet connects to the recess via the first channel. Thus, the virus sample can be dropped into the sample inlet and flow through the first channel to reach the recess and adhere onto the metal layer. Furthermore, the carrier may further comprise a second channel, the cover plate may comprise a gas outlet, and the gas outlet connects to the recess via the second channel. Thus, when a pump is connected to the gas outlet, the air between the carrier and the cover plate can be removed, and the virus sample can flow into the recess more efficiently.

In the present disclosure, the numbers of the recess, the first channel, the second channel, the sample inlet and the gas outlet are not particularly limited, and can be adjusted according to the need.

In the present disclosure, the metal layer may be made of gold, silver, copper, or an alloy thereof. For example, the metal layer is a gold layer.

In the present disclosure, a thickness of the metal layer may be in a range from 20 nm to 500 nm, for example, 40 nm to 500 nm, 60 nm to 500 nm, 80 nm to 500 nm, 100 nm to 500 nm, 120 nm to 500 nm, 140 nm to 500 nm, 160 nm to 500 nm, 180 nm to 500 nm, 200 nm to 500 nm, 220 nm to 500 nm, 240 nm to 500 nm, 260 nm to 500 nm, 280 nm to 500 nm or 300 nm to 500 nm.

In the present disclosure, a depth of the recess may be in a range from 0.1 mm to 20 mm, for example, 0.1 mm to 15 mm, 0.1 mm to 10 mm, 0.3 mm to 10 mm, 0.3 mm to 8 mm, 0.5 mm to 8 mm, 0.5 mm to 5 mm, 1 mm to 5 mm or 1 mm to 3 mm. However, the present disclosure is not limited thereto, and the depth of the recess can be adjusted according to the volume of the virus sample to be detected.

In the present disclosure, the area of the metal layer and the area of the recess are not particularly limited. The area of the metal layer corresponds to the area of the recess, so the metal layer can be placed into the recess well. In addition, the area of the recess also can be adjusted according to the volume of the virus sample to be detected.

The present disclosure also provides a detection system using the aforesaid detection device. The detection system of the present disclosure comprises: the aforesaid detection device; a Raman spectrometer providing an incident laser onto the metal layer of the detection device to obtain a Raman scattering signal; and an output device receiving the Raman scattering signal and outputting a Raman spectrum.

The present disclosure further provides a method for detecting viruses using the aforesaid detection device. The method of the present disclosure comprises the following steps: providing the aforesaid detection device and a Raman spectra virus database; applying a virus sample onto the plurality of cavities of the detection device; applying an incident light by a Raman spectrometer onto the metal layer of the detection device to generate a Raman spectrum of the virus sample; and comparing the Raman spectrum of the virus sample with a Raman spectra virus database to identify the species of the virus sample.

In the present disclosure, the wavelength of the incident light provided by the Raman spectrometer can be adjusted according to the size (for example, the depth) of the cavities or the viruses (for example, the size of the virus particle) to be detected. Thus, the optimized signal of the SERS effect can be obtained. In addition, the power of the incident light provided by the Raman spectrometer can be in a range from 1 mW to 5 mW, for example, 1.5 mW to 5 mW, 1.5 mW to 4.5 mW, 2 mW to 4.5 mW, 2.5 mW to 4.5 mW, 2.5 mW to 4 mW or 3 mW to 4 mW. If the power is too large, the sample may be degraded.

In the present disclosure, the species of the virus to be detected is not particularly limited. For example, the virus to be detected is SARS-CoV-2 virus or its variations.

It is known that the conventional detection method such as nucleic acid tests (for example, polymerase chain reaction (PCR) or reverse transcription PCR (RT-PCR)) or serological tests (for example, enzyme-linked immunosorbent assay (ELISA) or lateral flow immunoassay (LFIA)) is time-consuming or has its shortage. However, once the virus becomes a pandemic, a rapid and convincing detection system or method is required to timely identify the types of the virus in the patients, and thus the spread of the disease can be effectively controlled. Herein, the detection system and method of the present disclosure is performed by the SERS technology, so the types of the viruses can be identify rapidly and accurately. In addition, because the SERS effect can be improved by the nano-array structure on the metal layer in the detection device of the present disclosure, the types of the viruses still can be identify even though the concentration of the viruses in the virus sample is low.

The present disclosure further provides a method for preparing the aforesaid detection device. The method comprises the following steps: providing a mold, wherein a plurality of second protrusions are formed on a surface of the mold, and the plurality of second protrusions are arranged in an array; and applying the mold onto a metal layer to transfer a pattern of the plurality of second protrusions onto a first surface of the metal layer, wherein the metal layer is disposed in a recess of a carrier before or after the mold is applied onto the metal layer.

In the present disclosure, the detection device is prepared by using a mold. In particular, the detection device can be prepared through an imprint process by using the mold. Thus, the pattern of the second protrusion can be transferred onto the first surface of the metal layer by one step to form the cavities on the first surface of the metal layer. In the conventional process that the cavities have to be formed one by one, the process is time consuming and it is difficult to control the gap, depth or appearance of the cavities. In the method of the present disclosure, the cavities are prepared via the one-step imprint process by using the mold, so the cavities can be formed rapidly and the gap, depth or appearance of the cavities can be easily controlled.

In the present disclosure, a plasma treatment may be applied onto the first surface of the metal layer after the mold is applied onto the metal layer. Thus, the first surface of the metal layer can be more hydrophilic, and the virus sample can be easily adhered onto the first surface of the metal layer (in particular, the surface of the cavities). Herein, the plasma treatment may be held at low temperature, for example, ranging from 20° C. to 50° C., 25° C. to 50° C., 25° C. to 45° C., 30° C. to 45° C., 30° C. to 40° C. or 34° C. to 40° C. Herein, the plasma treatment may be held for 5 seconds to 300 seconds. In addition, the plasma treatment may be held with a power ranging from 1 W to 50 W, 15 W to 30 W or 17 W to 25 W. The plasma may be formed by a gas comprising He, Ar or a combination thereof. In addition, the gas may further comprise O₂, and the addition amount thereof may be ranged from 0.1 v/v % to 5 v/v %, or 0.1 v/v % to 2 v/v %. The gas may further comprise N₂, and the addition amount thereof may be ranged from 0.1 v/v % to 2 v/v %, or 0.1 v/v % to 1 v/v %. The flow of the gas may be ranged from 1 slm to 10 slm, or 1 slm to 5 slm. Furthermore, the working distance (i.e. the distance between the metal layer and the plasma) may be ranged from 1 mm to 12 mm.

Other novel features of the disclosure will become more apparent from the following detailed description when taken in conjunction with the accompanying drawings.

### BRIEF DESCRIPTION OF THE DRAWINGS

FIG. 1A is a cross-sectional view showing the preparation of a detection device according to Embodiment 1 of the present disclosure.

FIG. 1B is a perspective view showing a mold for preparing a detection device according to Embodiment 1 of the present disclosure.

FIG. 2 is a top view of a detection device according to Embodiment 1 of the present disclosure.

FIG. 3 is an exploded view of a detection device according to Embodiment 1 of the present disclosure.

FIG. 4 is a cross-sectional view of a detection device along the line A-A indicated in FIG. 2.

FIG. 5 is a top view of a microstructure on a metal layer of a detection device according to Embodiment 1 of the present disclosure.

FIG. 6 is a cross-sectional view of one cavity on a metal layer of a detection device according to Embodiment 1 of the present disclosure.

FIG. 7 is a cross-sectional view of a metal layer of a detection device according to Embodiment 2 of the present disclosure.

FIG. 8 is a SERS spectrum of SARS-CoV-2 pseudovirus using detection devices comprising metal layers with or without plasma treatment.

FIG. 9A is SERS spectra of SARS-CoV-2 pseudovirus taken at the same position of the array for 5 times.

FIG. 9B is a diagram showing the intensities of the SERS spectra of SARS-CoV-2 pseudovirus taken at the same position of the array for 5 times.

FIG. 10A is SERS spectra of SARS-CoV-2 pseudovirus taken at 6 different locations of the array.

FIG. 10B is a diagram showing the intensities of the SERS spectra of SARS-CoV-2 pseudovirus taken at 6 different locations of the array.

FIG. 11A is SERS spectra of SARS-CoV-2 pseudovirus, H1N1 and a combination thereof.

FIG. 11B is SERS spectra of SARS-CoV-2 pseudovirus, H3N2 and a combination thereof.

### DETAILED DESCRIPTION OF EMBODIMENT

Different embodiments of the present invention are provided in the following description. These embodiments are meant to explain the technical content of the present invention, but not meant to limit the scope of the present invention. A feature described in an embodiment may be applied to other embodiments by suitable modification, substitution, combination, or separation.

It should be noted that, in the present specification, when a component is described to have an element, it means that the component may have one or more of the elements, and it does not mean that the component has only one of the element, except otherwise specified.

Moreover, in the present specification, the ordinal numbers, such as “first” or “second”, are used to distinguish a plurality of elements having the same name, and it does not means that there is essentially a level, a rank, an executing order, or an manufacturing order among the elements, except otherwise specified. A “first” element and a “second” element may exist together in the same component, or alternatively, they may exist in different components, respectively. The existence of an element described by a greater ordinal number does not essentially means the existent of another element described by a smaller ordinal number.

Moreover, in the present specification, the terms, such as “top”, “bottom”, “left”, “right”, “front”, “back”, or “middle”, as well as the terms, such as “on”, “above”, “under”, “below”, or “between”, are used to describe the relative positions among a plurality of elements, and the described relative positions may be interpreted to include their translation, rotation, or reflection.

Moreover, in the present specification, when an element is described to be arranged “on” another element, it does not essentially means that the elements contact the other element, except otherwise specified. Such interpretation is applied to other cases similar to the case of “on”.

Moreover, in the present specification, the terms, such as “preferably” or “advantageously”, are used to describe an optional or additional element or feature, and in other words, the element or the feature is not an essential element, and may be ignored in some embodiments.

Moreover, in the present specification, when an element is described to be “suitable for” or “adapted to” another element, the other element is an example or a reference helpful in imagination of properties or applications of the element, and the other element is not to be considered to form a part of a claimed subject matter; similarly, except otherwise specified; similarly, in the present specification, when an element is described to be “suitable for” or “adapted to” a configuration or an action, the description is made to focus on properties or applications of the element, and it does not essentially mean that the configuration has been set or the action has been performed, except otherwise specified.

Moreover, in the present specification, a value may be interpreted to cover a range within ±10% of the value, and in particular, a range within ±5% of the value, except otherwise specified; a range may be interpreted to be composed of a plurality of subranges defined by a smaller endpoint, a smaller quartile, a median, a greater quartile, and a greater endpoint, except otherwise specified.

### Preparation Example—Preparation of Mold

The nano-array structure of the detection device of the present disclosure can be prepared by the nanoimprint process. The mold for nanoimprint process can be prepared by, for example, LIGA process, LIGA-like process, micromechanical machining, polymer micromachining, anisotropic etching, or focused ion beam (FIB). Herein, the LIGA process is briefly described below, but the present disclosure is not limited thereto.

A substrate is coated with a layer of a polymer resin material with a suitable thickness (for example, a few tens microns to a few hundreds of microns), and then a light source is applied onto the layer of the polymer resin material to develop the layer of the polymer resin material by using a photo mask. Thus, the pattern of the photo mask can be transferred to the layer of the developed polymer resin material to obtain a resin template. Next, a metal is deposited into the resin template by an electroforming process, following by removing the resin template via the etching process to obtain a metal mold insert with a microstructure.

Finally, a plastic mold for the sequential imprinting process can be obtained by a thermal imprint process or inject printing process with the metal mold insert, followed by removing the metal mold insert. Alternatively, the obtained plastic mold can further be used as a plastic mold insert, and a metal mold for the sequential imprinting process can be obtained by an electroforming process with the plastic mold insert, followed by removing the plastic mold insert.

After the aforesaid process, a plastic mold or a metal mold can be obtained, which can be used for forming the detection device of the present disclosure.

### Embodiment 1—Preparation of Detection Device

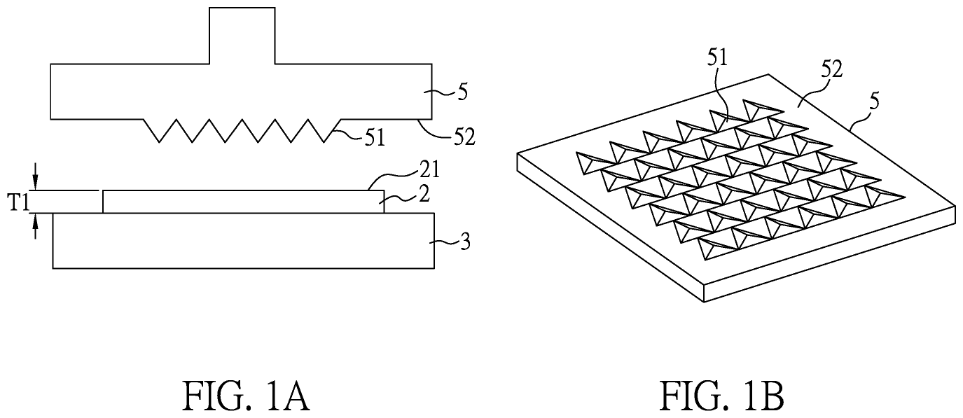

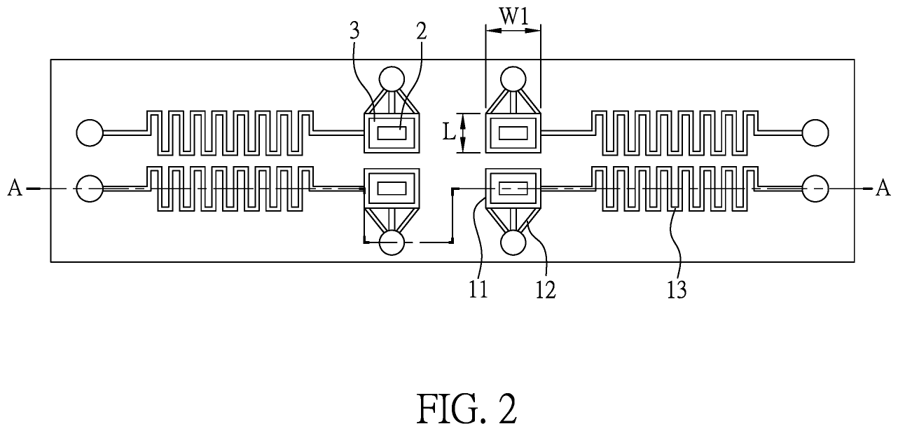

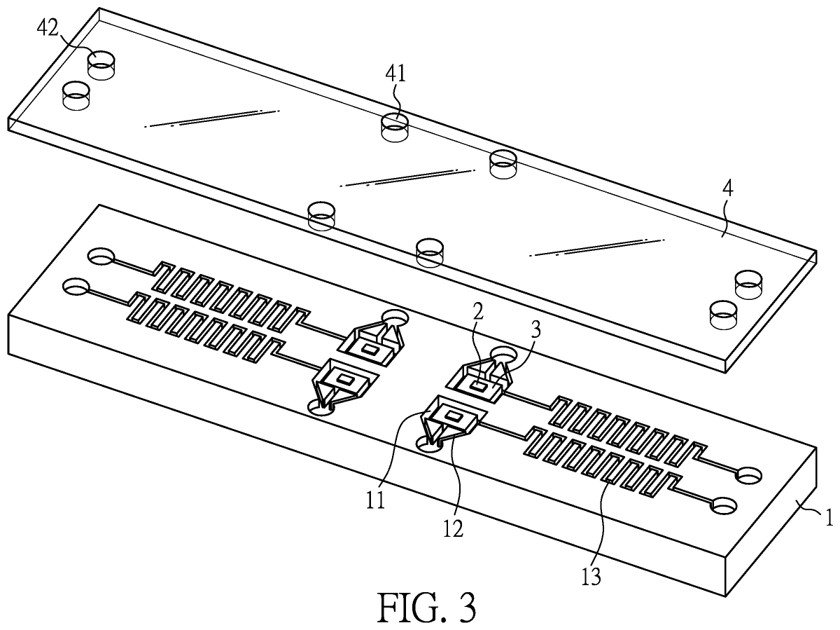

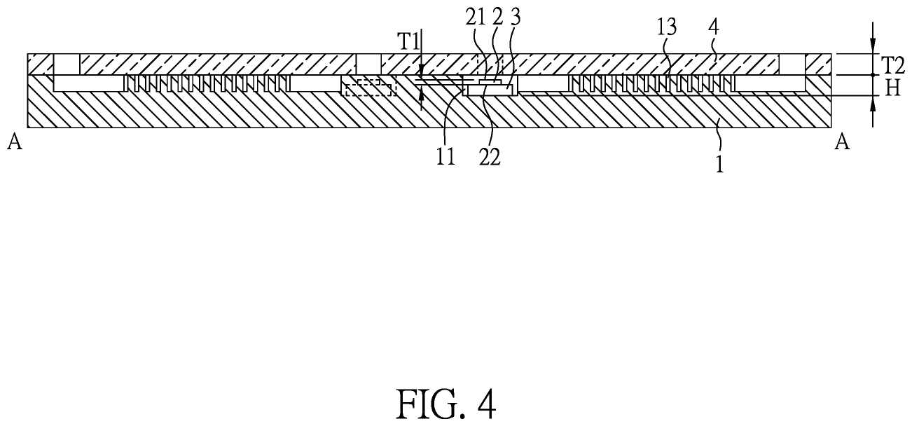

FIG. 1A is a cross-sectional view showing the preparation of a detection device according to Embodiment 1 of the present disclosure. FIG. 1B is a perspective view showing a mold for preparing a detection device according to Embodiment 1 of the present disclosure. FIG. 2 and FIG. 3 are respectively a top view and an exploded view of a detection device of the present embodiment. FIG. 4 is a cross-sectional view of the detection device along the line A-A indicated in FIG. 2.

As shown in FIG. 1A and FIG. 1B, a mold 5 prepared in Preparation example is provided, wherein a plurality of second protrusions 51 are formed on a surface 52 of the mold 5, and the second protrusions 51 are arranged in an array.

As shown in FIG. 2 to FIG. 4, a carrier 1 is provided, which comprises a recess 11. Herein, the carrier 1 can be a silicon substrate or a polymer substrate. In addition, the carrier 1 further comprises a first channel 12 and a second channel 13, respectively connecting to the recess 11. In the present embodiment, one recess 11 connects to three first channels 12 and one second channel 13, but the numbers of the first channel 12 and the second channel 13 connecting to one recess 11 is not limited thereto. In addition, the carrier 1 of the present embodiment comprises four recesses 11, but the number of the recess 11 is not limited thereto. The numbers of the recess 11, first channel 12 and the second channel 13 may be adjusted according to the need.

In the present embodiment, the height H, the width W1 and the length L of the recess 11 is not particularly limited, and may be adjusted according to the need. In one example, the height H may be 1 mm, the width W1 may be 5.25 mm and the length L may be 5.25 mm. Thus, the recess 11 can accommodate 5 μl to 20 μl of the virus liquid sample.

Next, a metal layer 2 is disposed on a substrate 3, wherein the metal layer 2 can be a gold foil and the substrate 3 can be a silicon substrate or a polymer substrate. Herein, the thickness T1 of the metal layer 2 may be ranged from 20 nm to 500 nm. In the present embodiment, the thickness T1 of the metal layer 2 is 200 nm. Then, the mold 5 is placed onto the metal layer 2, and a predetermined force is applied onto the mold 5 to perform the nanoimprint process. After the nanoimprint process, the pattern of the second protrusions 51 of the mold 5 can be transferred on to a first surface 21 of the metal layer 2, so a microstructure can be formed on the first surface 21 of the metal layer 2.

Then, the substrate 3 disposed with the metal layer 2 having the microstructure is placed into the recess 11. The metal layer 2 is treated with plasma to increase the hydrophilicity of the metal layer 2. Herein, the argon plasma source is used to treat the metal layer 2 for 10 seconds (input power of 5 W, gas flow rate of 2 slm), and a distance between the nozzle and the metal layer 2 is 8 mm. Before the plasma treatment, the water-air contact angle of the metal layer 2 is ranged from 60 degrees to 70 degrees. After the plasma treatment, the water-air contact angle of the metal layer 2 is ranged from 0 degrees to 10 degrees, in particular, less than 10 degrees. The condition for the plasma treatment is not limited to that illustrated above, and can be adjusted according to the material of the metal layer 2 or the desired contact angle of the metal layer 2.

After the plasma treatment, a cover plate 4 is placed onto the carrier 1. Herein, the cover plate 4 comprises a sample inlet 41 and a gas outlet 42. The cover plate 4 has a transmittance of more than 89%, and the material of the cover plate 4 may be a polymer substrate such as a PMMA substrate. In addition, the cover plate 4 may have a thickness T2 of 0.05 mm to 0.3 mm.

After the aforesaid process, the detection device of the present embodiment is accomplished. The detection device comprises: a carrier 1 comprising a recess 11; and a metal layer 2 disposed in the recess 11 and having a contact angle ranging from 0 degrees to 10 degrees. In addition, the detection device further comprises a substrate 3, wherein the substrate 3 is disposed in the recess 11 and the metal layer 2 is disposed on the substrate 3. Furthermore, the detection device further comprises a cover plate 4 disposed on the metal layer 2. The carrier 1 further comprises a first channel 12, the cover plate 4 comprises a sample inlet 41, and the sample inlet 41 connects to the recess 11 via the first channel 12. The carrier 1 further comprises a second channel 13, the cover plate 4 comprises a gas outlet 42, and the gas outlet 42 connects to the recess 11 via the second channel 13. When a virus sample is loaded into the sample inlet 41, the virus sample can flow through the first channel 12 to reach the recess 11 and adhere onto the metal layer 2. In addition, when a pump (not shown in the figure) is connected to the gas outlet 42, the air between the carrier 1 and the cover plate 4 can be removed, and the virus sample can flow into the recess 11 more efficiently. Hereinafter, the microstructure on the metal layer 2 is described below.

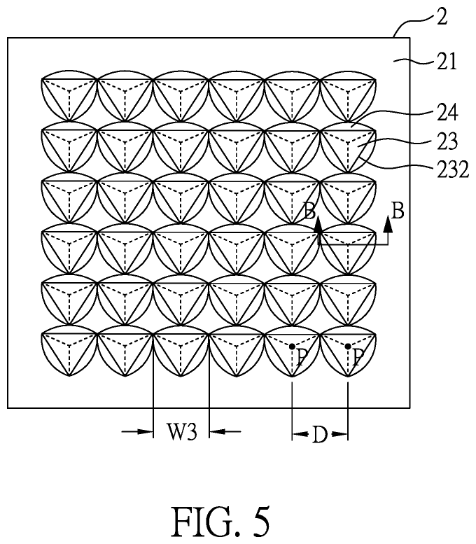

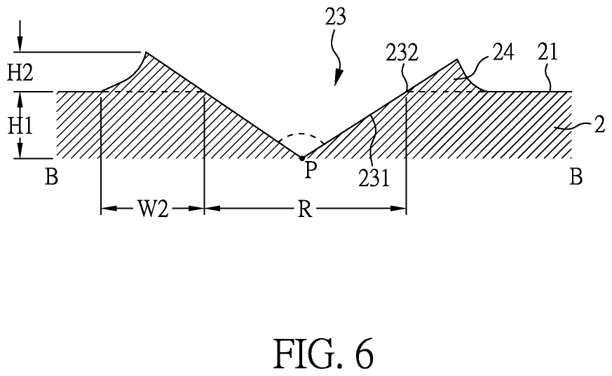

FIG. 5 is a top view of a microstructure on the metal layer, and FIG. 6 is a cross-sectional view of one cavity on the metal layer alone the line B-B indicated in FIG. 5.

As shown in FIG. 4 to FIG. 6, in the detection device of the present embodiment, a plurality of cavities 23 are formed on a first surface 21 of the metal layer 2 opposite to a second surface 22 of the metal layer 2 facing the carrier 1, the cavities 23 are arranged in an array, and a plurality of first protrusions 24 are formed on the first surface 21 of the metal layer 2 and near to the cavities 23.

Herein, the cavities 23 are arranged in a 6×6 array, but the present disclosure is not limited thereto. In other embodiments of the present disclosure, the cavities 23 may be arranged in an n×m array, wherein n and m are respectively an integral of 1 or more. In addition, in the present embodiment, one array is formed on the first surface 21 of the metal layer 2, but the present disclosure is not limited thereto. In other embodiments of the present disclosure, plural arrays may be formed on the first surface 21 of the metal layer 2. When plural arrays are formed, these arrays can be formed by one imprint step with plural molds, or by plural imprint steps with one mold, or by plural imprint steps with plural molds.

In the present embodiment, each of the cavities 23 has a shape of an inverted polygonal pyramid. Herein, each of the cavities 23 has a shape of an inverted equilateral triangular pyramid, but the present disclosure is not limited thereto. In addition, each of the cavities 23 has a bottom point P, and a distance D between the bottom points P of two adjacent cavities 23 is in a range from 100 nm to 1000 nm. This distance D may be adjusted according to the need.

Furthermore, each of the cavities 23 has a plurality of inclined surfaces 231, and one of the first protrusions 24 connects to one of the inclined surfaces 231. In the present embodiment, the cavity 23 has a triangular opening R with three sides 232 which respectively connects to one of the first protrusions 24, and thus one first protrusion 24 connects to one inclined surface 231 at each side 232 of the triangular opening R. Herein, the triangular opening R of the cavity 23 can be defined by the first surface 21 of the metal layer 2 and/or the extension surface (as indicated by the dash lines) of the first surface 21, and the side 232 of the cavity 23 is defined by the first surface 21 of the metal layer 2 and/or the extension surface of the first surface 21.

In the present embodiment, each of the cavities 23 respectively has a depth H1 ranging from 20 nm to 300 nm and a width W3 ranging from 600 nm to 1400 nm measured from the first surface 21 of the metal layer 2. In addition, each of the first protrusions 24 respectively has a height H2 ranging from 2 nm to 60 nm and a width W2 ranging from 270 nm to 500 nm measured from the first surface 21 of the metal layer 2. The depth H1, the height H2, the width W2 and the width W3 may be varied according to the virus to be detected. In addition, the depth H1, the height H2, the width W2 and the width W3 may be adjusted by, for example, the loading applied onto the mold 5 (as shown in FIG. 5) or the lasting time of the imprinting step. For example, when the same mold 5 is used, the depth H1, the height H2, the width W2 and the width W3 of the cavities 23 and the first protrusions 24 in one array may be different from the depth H1, the height H2, the width W2 and the width W3 of the cavities 23 and the first protrusions 24 in another array by changing the loading applied onto the mold 5 or the lasting time of the imprinting step.

In the present embodiment, the metal layer 2 is disposed on the substrate 3. In another embodiment of the present disclosure, the metal layer 2 may be directly disposed in the recess 11 without using the substrate 3. In this case, the metal layer 2 may be placed into the recess 11 first, and then the imprint process is applied onto the metal layer 2.

### Embodiment 2—Detection Device

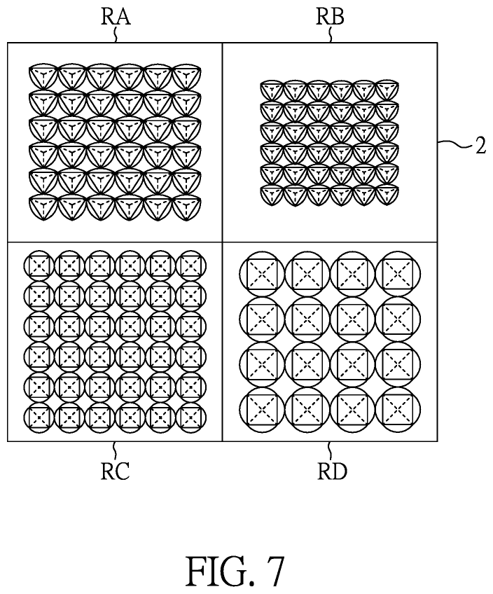

FIG. 7 is a cross-sectional view of a metal layer of a detection device according to Embodiment 2 of the present disclosure.

The process for preparing the detection device is similar to that shown in Embodiment 1, except that four different arrays are formed on the metal layer 2. Thus, the metal layer 2 of the present embodiment comprises four regions RA, RB, RC and RD.

The array in the region RA is similar to that shown in FIG. 5. The array in the region RB is similar to that in the region RA, but the sizes of the cavities and first protrusions are smaller than those in the region RA. The array in the region RC is similar to that in the region RA, but each of the cavities 23 respectively has a shape of an inverted rectangular pyramid. The array in the region RD is similar to that in the region RC, but the sizes of the cavities and first protrusions are greater than those in the region RC and the array is a 4×4 array.

Herein, the arrays in the regions RA and RB may be formed by different molds, or the arrays in the regions RA and RB may be formed by the same mold by changing the loading applied onto the mold or the lasting time of the imprinting step. In addition, the arrays in the regions RA, RB, RC and RD may be formed sequentially or at the same time.

In the present embodiment, one metal layer 2 has four regions RA, RB, RC and RD with different arrays, so it is possible to detect different viruses in one virus sample at the same time.

The numbers of the arrays and the shapes and sizes of the cavities are not limited to those shown in FIG. 7, and may be adjusted according to the need.

### Embodiment 3—Detection System

The detection device of Embodiment 1 or 2 may be used with a Raman spectrometer and an output device to form a detection system. Herein, the Raman spectrometer can provide an incident laser onto the metal layer of the detection device to obtain a Raman scattering signal, and the output device can receive the Raman scattering signal and outputting a Raman spectrum.

### Testing Example

The detection device of Embodiment 1 and the detection system of Embodiment 3 is used in the present texting example. The procedure for detecting a virus comprises the following steps: providing a detection device and a Raman spectra virus database; applying a virus sample onto the plurality of cavities of the detection device; applying an incident light by a Raman spectrometer onto the metal layer of the detection device to generate a Raman spectrum of the virus sample; and comparing the Raman spectrum of the virus sample with a Raman spectra virus database to identify the species of the virus sample.

### Preparation of SARS-CoV-2 Pseudovirus

SARS-CoV-2 pseudovirus was produced following the process done by Huang et al. (Huang, S. W., Tai, C. H., Hsu, Y. M., Cheng, D., Hung, S. J., Chai, K. M., Wang, Y. F., Wang, J. R., 2020. Assessing the application of a pseudovirus system for emerging SARS-CoV-2 and re-emerging avian influenza virus H5 subtypes in vaccine development. Biomed. J. 43, 375-387). The lentiviral vector system was provided by the National RNAi Core of Academia Sinica Taiwan. De novo synthesis was performed to obtain sequences of the spike protein, which were then cloned into the pMD.G plasmid to express SARS-CoV-2 pseudoviruses. Cells were transfected with 1 μg pCMVdeltaR8.91, pLAS2w.RFP-C.Pneo and pMD.G plasmids; pMD.G with S gene for SARS-CoV-2 was tagged by hemagglutinin (HA) on the C-terminus. Rabbit polyclonal antibody against SARS-CoV S-protein (ARG54885, Arigo Biolaboratories) and mouse anti-HA tag monoclonal antibody (C05012-100UG, Croyez Bio) were used to produce the SARS-CoV-2 pseudovirus.

### Preparation of H1N1 Virus and H3N2 Virus

The samples containing H1N1 or H3N2 virus used in the present embodiment was provided by Dr. Wang, Jen-Ren in Department of Medical Laboratory Science and Biotechnology, College of Medicine, National Cheng Kung University, and the virus stains were obtained from Department of Pathology, National Cheng Kung University Hospital. MOCK cells (Madin-Darby Canine Kidney cells) were cultured in 75T culture flasks. After a full monolayer of cells was obtained, the original medium was removed, and the cells were washed with PBS buffer twice. After removing the PBS buffer, the virus fluid was placed into a 37° C. water bath to thaw quickly. To infect the cells, the thawed cells were added into a virus culture tube with a single cell layer, followed by shaking evenly, so that the cell surfaces can completely contact with the virus liquid. Then, the culture was placed into an incubator at 35° C. for 1 hour. After adding suitable amount of the culture containing influenza virus, the obtained culture was again placed into the incubator at 35° C. When 75% of the cells had the cytopathic effect (CPE), the viruses were divided into portions and stored.

### Detection

Herein, the detection device prepared in Embodiment 1 was used. The used detection device has the metal layer having the microstructure shown in FIG. 5 and FIG. 6. In one example, the distance D between the bottom points P of two adjacent cavities 23 is 520 nm and the depth H1 of the cavities 23 is 100 nm. In another example, the distance D between the bottom points P of two adjacent cavities 23 is 600 nm and the depth H1 of the cavities 23 is 120 nm. In further another example, the distance D between the bottom points P of two adjacent cavities 23 is 700 nm and the depth H1 of the cavities 23 is 140 nm.

The SERS experiments were performed with the use of the Raman spectrometer (UniDRON, CL Technology Co. Ltd.) with a laser source of 633 nm and a maximum laser power of 35 mW. The pseudovirus sample was dropped onto the detection device before subjecting to the Raman laser for measurement. In this experiment, spectra were taken 5 times on the same spot on the detection device to investigate the effect of exposing the same area on the change of peak intensities, and 6 different locations on the same detection device were tested to see the reproducibility of results and consistency of the SERS spectra. In addition, the laser power of 3.5 mW was used for measurement.

### Results

Herein, the results of the detection device with the microstructure having cavities with the distance D of 600 nm and the depth H1 of 120 nm (as shown in FIG. 5 and FIG. 6) are present below.

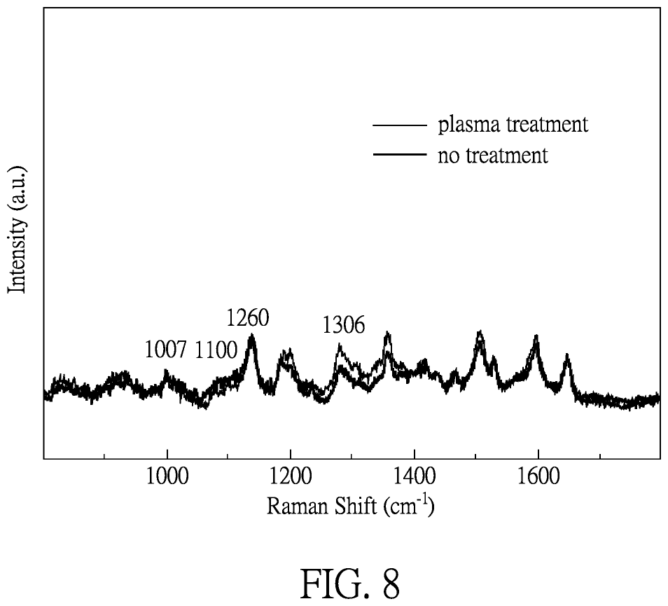

FIG. 8 is a SERS spectrum of SARS-CoV-2 pseudovirus using detection devices comprising metal layers with or without plasma treatment. Compared to the detection device in which the metal layer is not treated with plasma and has the contact angle of 60-70 degrees, the spectrum of the SARS-CoV-2 pseudovirus measured by using the detection device in which the metal layer is treated with plasma shows four enhanced peaks which are respectively 1007, 1100, 1260 and 1306 cm⁻¹. This result indicated that the intensities of the Raman peaks can further be enhanced by increasing the hydrophilicity of the metal layer with plasma treatment.

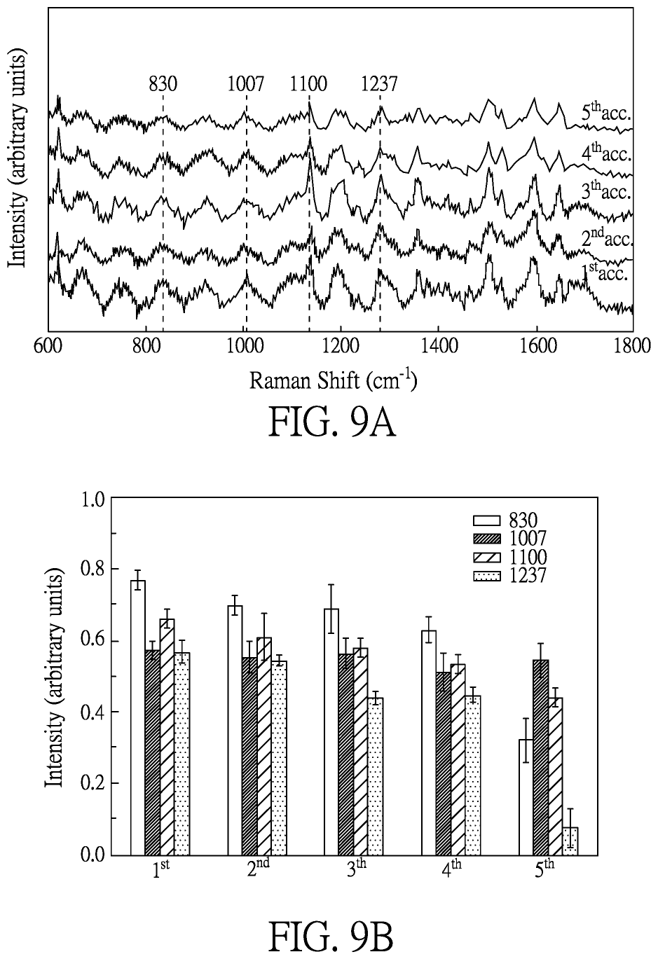

FIG. 9A is SERS spectra of SARS-CoV-2 pseudovirus taken at the same position of the array for 5 times, and FIG. 9B is a diagram showing the intensities of the SERS spectra of SARS-CoV-2 pseudovirus taken at the same position of the array for 5 times. These results indicate that the same characteristic peaks 830, 1007, 1100, and 1237 cm⁻¹ are found at the same Raman shifts throughout the 5 measurements but with a decline in intensities with the increasing use of the same spot (from 1^{st} to 5^{th} accumulation). This decline in intensities is due to the damage caused by the laser to both the array and the analytes by the continuous use of the same spot.

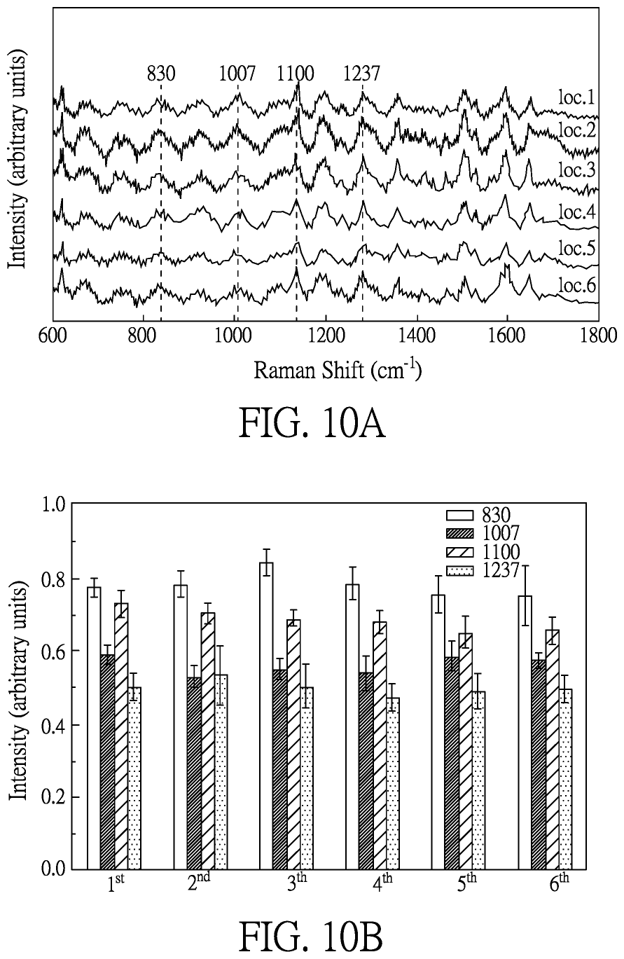

FIG. 10A is SERS spectra of SARS-CoV-2 pseudovirus taken at 6 different locations of the array, and FIG. 10B is a diagram showing the intensities of the SERS spectra of SARS-CoV-2 pseudovirus taken at 6 different locations of the array. These results indicate that characteristic peaks as marked FIG. 10A are strong regardless of location, but it should be noted that some peaks are not as apparent in some locations. This is most likely due to the non-uniformity of microstructures in the array.

From the results shown in FIG. 9A to FIG. 10B, four characteristic peaks about 830, 1007, 1100, and 1237 cm⁻¹ are found at the same Raman shifts in the SERS spectra of SARS-CoV-2 pseudovirus.

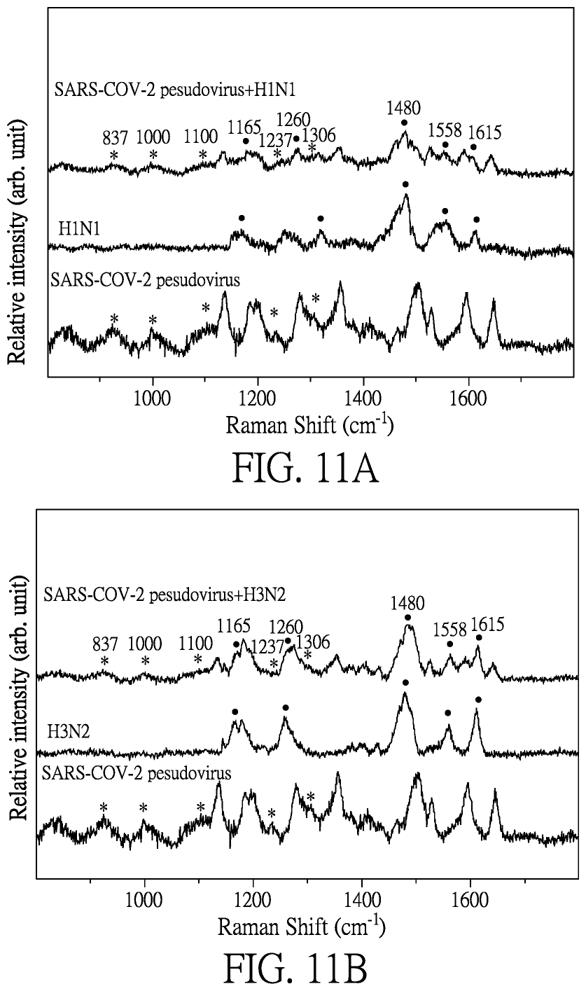

FIG. 11A is SERS spectra of SARS-CoV-2 pseudovirus, H1N1 and a combination thereof, and FIG. 11B is SERS spectra of SARS-CoV-2 pseudovirus, H3N2 and a combination thereof. As shown in FIG. 11A and FIG. 11B, when a sample containing multiple viruses (SARS-CoV-2 pseudovirus and H1N1 in FIG. 11A, and SARS-CoV-2 pseudovirus and H3N2 in FIG. 11B), it is possible to detect the multiple viruses at the same time when using the detection device of the present disclosure.

When a virus sample is detected, the intensities of the SERS spectra can be taken at plural locations of the array. When the characteristic peaks of the virus to be detected are found, it means that the virus sample contains the target virus particles. In addition, by analyzing the percentage of the locations that the characteristic peaks of the virus are found, it is possible to know the relative amount of the virus particles contained in the virus sample.

In the present disclosure, the array of the cavities is formed by an imprint process, so the gaps (i.e. the distance between two bottom tips of two adjacent cavities), shapes, depths and appearance of the cavities can be easily controlled. Thus, the detection device can be prepared in a rapid and simple way. In addition, when a virus sample is loaded into the detection device, the virus particles can be entrapped into the cavities formed on the metal layer. When a Raman spectrometer is used to detect the virus sample with light having specific wavelength (for example, 633 nm or 785 nm), hot spots are generated under the virus particles and the strongest hot spots are appeared at bottom points of the cavities. Thus, the Raman signals of the virus particles can be enhanced. Furthermore, when the metal layer of the detection device is treated with plasma, the hydrophilicity of the metal layer can be enhanced. Thus, the virus sample can be easily adhered onto the surface of the metal layer, the virus particles can be easily entrapped into the cavities, the Raman signals can be enhanced, and the detection noise can be reduced.

Although the present disclosure has been explained in relation to its embodiment, it is to be understood that many other possible modifications and variations can be made without departing from the spirit and scope of the disclosure as hereinafter claimed.

What is claimed is:

1. A detection device, comprising:
a carrier comprising a recess; and
a metal layer disposed in the recess and having a contact angle ranging from 0 degrees to 10 degrees,
wherein a plurality of cavities are formed on a first surface of the metal layer opposite to a second surface of the metal layer facing the carrier, the plurality of cavities are arranged in an array, and a plurality of first protrusions are formed on the first surface of the metal layer and near to the plurality of cavities.

2. The detection device of claim 1, further comprising a substrate, wherein the substrate is disposed in the recess and the metal layer is disposed on the substrate.

3. The detection device of claim 1, wherein each of the plurality of cavities has a shape of an inverted polygonal pyramid.

4. The detection device of claim 3, wherein each of the plurality of cavities has a bottom point, and a distance between the bottom points of two adjacent cavities of the plurality of cavities is in a range from 100 nm to 1000 nm.

5. The detection device of claim 3, wherein each of the plurality of cavities has a plurality of inclined surfaces, and one of the plurality of first protrusions connects to one of the plurality of inclined surfaces.

6. The detection device of claim 1, wherein each of the plurality of cavities has a depth ranging from 20 nm to 300 nm measured from the first surface of the metal layer.

7. The detection device of claim 1, wherein each of the plurality of first protrusions has a height ranging from 2 nm to 60 nm measured from the first surface of the metal layer.

8. The detection device of claim 1, further comprising a cover plate disposed on the metal layer.

9. The detection device of claim 8, wherein the cover plate has a transmittance more than 89%.

10. The detection device of claim 8, wherein the carrier further comprises a first channel, the cover plate comprises a sample inlet, and the sample inlet connects to the recess via the first channel.

11. The detection device of claim 8, wherein the carrier further comprises a second channel, the cover plate comprises a gas outlet, and the gas outlet connects to the recess via the second channel.

12. The detection device of claim 1, wherein the metal layer is made of gold, silver, copper, or an alloy thereof.

13. A detection system, comprising:
a detection device, comprising:
a carrier comprising a recess; and
a metal layer disposed in the recess and having a contact angle ranging from 0 degrees to 10 degrees,
wherein a plurality of cavities are formed on a first surface of the metal layer opposite to a second surface of the metal layer facing the carrier, the plurality of cavities are arranged in an array, and a plurality of first protrusions are formed on the first surface of the metal layer and near to the plurality of cavities;
a Raman spectrometer providing an incident laser onto the metal layer of the detection device to obtain a Raman scattering signal; and
an output device receiving the Raman scattering signal and outputting a Raman spectrum.

14. A method for detecting viruses, comprising the following steps:
providing a detection device and a Raman spectra virus database, wherein the detection device comprises:
a carrier comprising a recess; and
a metal layer disposed in the recess and having a contact angle ranging from 0 degrees to 10 degrees,
wherein a plurality of cavities are formed on a first surface of the metal layer opposite to a second surface of the metal layer facing the carrier, the plurality of cavities are arranged in an array, and a plurality of first protrusions are formed on the first surface of the metal layer and near to the plurality of cavities;
applying a virus sample onto the plurality of cavities of the detection device;
applying an incident light by a Raman spectrometer onto the metal layer of the detection device to generate a Raman spectrum of the virus sample; and
comparing the Raman spectrum of the virus sample with a Raman spectra virus database to identify the species of the virus sample.

15. A method for preparing the detection device, comprising the following steps:
providing a mold, wherein a plurality of second protrusions are formed on a surface of the mold, and the plurality of second protrusions are arranged in an array; and
applying the mold onto a metal layer to transfer a pattern of the plurality of second protrusions onto a first surface of the metal layer,
wherein the metal layer is disposed in a recess of a carrier before or after the mold is applied onto the metal layer,
wherein the metal layer has a contact angle ranging from 0 degrees to 10 degrees,
wherein a plurality of cavities are formed on the first surface of the metal layer opposite to a second surface of the metal layer facing the carrier, the plurality of cavities are arranged in an array, and a plurality of first protrusions are formed on the first surface of the metal layer and near to the plurality of cavities.

16. The method of claim 15, wherein a plasma treatment is applied onto the first surface of the metal layer after the mold is applied onto the metal layer.

17. The method of claim 16, wherein a temperature of the plasma treatment is ranged from 20° C. to 50° C.

18. The method of claim 16, wherein the plasma treatment is applied for 5 seconds to 300 seconds.

19. The method of claim 15, wherein each of the plurality of cavities has a shape of an inverted polygonal pyramid.

20. The method of claim 19, wherein each of the plurality of cavities has a bottom point, and a distance between the bottom points of two adjacent cavities of the plurality of cavities is in a range from 100 nm to 1000 nm.
